(19) United States
(12) Patent Application Publication
(10) Pub. No.: US 2016/0213444 A1
(43) Pub. Date: Jul. 28, 2016

(54) DEVICE AND METHOD FOR SANITIZING GAS AND WATER

(71) Applicant: Kirkor Kiremitci, Laval (CA)

(72) Inventor: Kirkor Kiremitci, Laval (CA)

(21) Appl. No.: 15/007,548

(22) Filed: Jan. 27, 2016

Related U.S. Application Data
(63) Continuation-in-part of application No. 14/606,541, filed on Jan. 27, 2015.

(51) Int. Cl. A61C 1/00; C02F 1/32; A61L 2/10

(52) U.S. Cl. CPC A61C 1/0076; A61L 2/10; C02F 1/32; A61C 1/0092; C02F 2103/006

(57) ABSTRACT

A device for preventing build up and/or reducing biofilm pathogens from medical or dental fluids includes a housing defining a sanitizing chamber. A water conduit may extend through the sanitizing chamber and channel water from a water inlet to a water outlet. A gas inlet may receive pressurized gas and gas outlet may flow the pressurized gas from the inlet to the gas outlet through the sanitizing chamber. A light source emits light into the sanitizing chamber, the emitted light being incident on the water conduit and/or the pressurized gas in the sanitizing chamber and having wavelengths effective for removing biofilm pathogens from the water and/or the pressurized gas to sanitize the water and the pressurized gas. The sanitized water and gas may be used when performing medical or dental procedures. The device may further include a recirculation system for recirculating water from the water outlet to the water inlet.

### TECHNICAL FIELD

The present subject-matter relates to devices and methods for sanitizing gas and water, and more particularly to devices and methods for sanitizing gas and water within a sanitizing chamber.

### INTRODUCTION

Various fields make use of a source of water and/or a source of gas. Tools used in such fields may be powered by water from the source of water or gas from the source of gas. Tools used may also dispense water and/or gas. Such fields may include the medical fields and dental field, where the water and/or gas may be used or dispensed when performing various dental or medical procedures.

The source of water may be stored or treated and fed to a water-based tool via a water line. The storage mechanism or treating mechanism may be located in a location that is remote of the location of the water-based tool. In some situations, the water line may have a substantial length.

Similarly, the source of gas may be stored or treated and is fed to the gas-based tool a gas line. Typically, the storage mechanism or the treating mechanism may be located in a location that is remote of the location of the gas-based tool.

Microorganisms which may be harmful may be present in the source of water and/or source of gas or may be introduced in various ways. In the case of water, the source of water may be water from a water utility. Alternatively, the source of water may be sterilized water. Such sources may already contain microorganisms or travel through systems that contain microorganisms. Furthermore, microorganisms may build up over time, creating, for example, biofilm in the water line for transporting water from the water source to the water-based tool.

In the case of pressurized gas, the gas may be compressed by an air compressor. This air compressor may be located remotely from the location of the gas-based tool. This may be done to reduce noise and/or vibration created by the air compressor. Accordingly, the air compressor may be located in an area that supports growth of microorganisms. Furthermore, compressor tanks contain some humidity due to condensation, which further can promote microorganism growth.

Filters may be provided within the gas compressor, but require proper maintenance. Moreover, like water systems, microorganisms, such as in a biofilm, may build up over time in the gas line for transport from the gas source to the gas-based tool. Even where filters perform adequately, the gas line may represent a potential source of microorganisms.

A drying system may be further provided in a gas system, which may further cause build-up of microorganisms, such as biofilm, in the gas transport system.

U.S. publication number 2012/0241644 to Ben-David et al. discloses a water purification apparatus, comprising an elongate UV source and a conduit for water to be purified formed of a UV transmissive material, wherein the conduit has an inlet and an outlet and positioned so that part of it is wrapped around at least part of the UV source thereby to sterilize water within the conduit, further comprising reflective means for UV radiation to be reflected onto one or more parts of the conduit which extends beyond the part which is wrapped around the UV source.

U.S. Pat. No. 6,464,868 to Korin discloses a method for removing biofilm from, and/or for preventing biofilm from forming on, an interior surface of a conduit that receives a supply of water, is performed by disabling the supply of water to the conduit, and passing an ozone-containing gas to the conduit. The ozone-containing gas can be generated from an oxygen-containing gas that is exposed to either a corona discharge or ultraviolet radiation. In an alternate embodiment, the water is disinfected by the ultraviolet radiation.

U.S. Pat. No. 5,935,431 to Korin discloses an integrated filtration and sterilization apparatus including an ultraviolet lamp disposed within an ultraviolet radiation permeable sleeve such that a gas conduit is formed between an outer surface of the ultraviolet lamp and an inner surface of the sleeve; a filtration member disposed about the sleeve such that a permeate chamber is formed between an outer surface of the sleeve and an inner surface of the filtration member; a feed chamber disposed about an outer surface of the filtration member; a liquid feed for feeding a liquid to the feed chamber and a liquid removal line for removing liquid from the permeate chamber; a gas feed for feeding an oxygen-containing gas to the gas conduit and a gas removal line for removing an ozone-containing gas from the gas conduit; and a mixing device for mixing liquid removed from the permeate chamber and ozone-containing gas from the gas conduit downstream from the permeate chamber.

U.S. publication number 2003/0190254 to Falat discloses an ultraviolet apparatus or system for delivering substantially pure compressed air to desired locations. The invention is particularly useful for delivering substantially pure compressed air to dental unit hand pieces and instruments. The invention provides a dental chair unit including apparatus for delivering substantially pure 99.9% compressed air and method for delivering substantially pure compressed air to a dental hand pieces, other medical applications and also in industrial automotive assemblies with pneumatic tools.

### SUMMARY

It would thus be highly desirable to be provided with a system or method that would at least partially address the disadvantages of the existing technologies.

The embodiments described herein provide in one aspect a device for preventing build up and/or reducing biofilm pathogens from medical or dental pressurized gas and water, the device comprising a housing defining a sanitizing chamber, a water conduit extending through the sanitizing chamber, the water conduit channeling water from a water inlet to a water outlet, a gas inlet for receiving pressurized gas, a gas outlet, the pressurized gas flowing from the inlet to the gas outlet through the sanitizing chamber, a light source for emitting light into the sanitizing chamber, the emitted light being incident on the water conduit and the pressurized gas in the sanitizing chamber and having wavelengths effective for removing biofilm pathogens from the water and the pressurized gas to sanitize the water and the pressurized gas. The sanitized water and gas is used when performing medical or dental procedures.

The embodiments described herein provide in another aspect a device for preventing build up and/or reducing biofilm pathogens from pressurized gas used in a medical or dental surgical tool, the device comprising: a housing defining a sanitizing chamber, a gas inlet for receiving pressurized gas, a gas outlet, the pressurized gas flowing from the gas inlet to the gas outlet through the sanitizing chamber, a light source for emitting light into the sanitizing chamber, the emitted light being incident on the pressurized gas in the sanitizing chamber and having wavelengths effective for removing biofilm pathogens from the pressurized gas to sanitize the water and the pressurized gas, and at least one member disposed in the sanitizing chamber for causing turbulence in the pressurized gas within the sanitizing chamber. The sanitized gas is used when performing medical or dental procedures.

The embodiments described herein provide in yet another aspect a device for preventing build up and/or reducing biofilm pathogens from medical or dental fluid, the device comprising a housing defining a sanitizing chamber; an inlet for receiving one of pressurized gas and water, an outlet, said one of the pressurized gas and water flowing from the inlet to the outlet through the sanitizing chamber, a light source for emitting light into the sanitizing chamber, the emitted light being incident on said one of the pressurized gas and water gas and having wavelengths effective for removing biofilm pathogens from said one of the pressurized gas and water to sanitize the water or the pressurized gas, an ultra-violet sensor for measuring an ultra-violet light level within the sanitizing chamber, an alarm, and a controller for receiving the measured ultra-violet light level and controlling the alarm to emit an alarm indicator when the measured ultra-violet light level falls below a predetermined ultra-violet light threshold. The sanitized water or gas is used when performing medical or dental procedures.

The embodiments described herein provide in yet another aspect a device for sanitizing, the device comprising a recirculation system including a recirculation return tube for recirculating water from the water outlet to the water inlet, a water source connection fluidly connecting a water source and the recirculation return tube to the water inlet, and a tool connection fluidly connecting a water control circuit outlet to a tool joint and the recirculation return tube. The tool joint is connectable to a medical or dental surgical tool that uses the sanitized water and/or pressurized gas.

In an aspect, the recirculation includes a recirculation pump for pumping water in the recirculation return tube. In an aspect, the recirculation includes a flow sensor to determine water flow in the recirculation return tube, wherein when the determined flow is below a predetermined level, the recirculation system will trigger an alarm. In an aspect, the recirculation includes a one way check valve in the recirculation return tube to prevent backflow of water in the recirculation return tube.

Other aspects and features will become apparent, to those ordinarily skilled in the art, upon review of the following description of some exemplary embodiments.

### BRIEF DESCRIPTION OF THE DRAWINGS

The drawings included herewith are for illustrating various examples of articles, methods, and apparatuses of the present specification. In the drawings:

FIG. 1 illustrates a perspective view of a sanitizing device for sanitizing water and pressurized gas according to one exemplary embodiment;

FIG. 2 illustrates a cross-sectional view along the line A-A of the exemplary sanitizing device 1 wherein various elements are not shown;

FIG. 3 illustrates a cross-sectional view of a region of a first housing of the exemplary sanitizing device in proximity of a first end opening;

FIG. 4 illustrates a cross-sectional view of a lighting mechanism according to one exemplary embodiment;

FIG. 5 illustrates a cross-sectional view of the exemplary sanitizing device having the lighting mechanism fitted in its sanitizing chamber;

FIG. 6 illustrates a cross-sectional view along the line B-B of the exemplary sanitizing device;

FIG. 7 illustrates a cross-sectional view of the exemplary sanitizing device having a water conduit;

FIG. 8 illustrates a cross-sectional view along the line C-C of the exemplary sanitizing device;

FIG. 9 illustrates a plan view of the sanitizing device according to one exemplary embodiment;

FIG. 10 illustrates a block diagram of a system for controlling sanitization of the sanitizing device according to one exemplary embodiment; and

FIG. 11 illustrates a block diagram of a recirculation system, in accordance with an embodiment.

### DETAILED DESCRIPTION

Various apparatuses or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below. It is possible that an apparatus or process described below is not covered by any of the claimed embodiments. Any embodiment disclosed below that is not claimed in this document may be the subject matter of another protective instrument, for example, a continuing patent application, and the applicants, inventors or owners do not intend to abandon, disclaim or dedicate to the public any such embodiment by its disclosure in this document.

According to various exemplary devices described herein, the pressurized gas remains pressurized while flowing through the sanitizing chamber.

According to various exemplary devices described herein, the water flowing through the water conduit is physically separated from the pressurized gas flowing through the sanitizing chamber.

According to various exemplary devices described herein, the sanitizing chamber is elongated and the light source extends along a portion of a length of the elongated sanitizing chamber, the light being emitted in a radial direction of the sanitizing chamber.

According to various exemplary devices described herein, the water conduit is coiled about a first portion of the light source and at least a second portion of the light source is free of the coiled water conduit.

According to various exemplary devices described herein, at least a quarter of the length of the light emitting source is free of the coiled water conduit.

According to various exemplary devices described herein, the devices further include at least one member disposed in the sanitizing chamber for causing turbulence in the pressurized gas within the sanitizing chamber.

According to various exemplary devices described herein, the at least one member comprises a baffle plate positioned in proximity of the gas inlet, the baffle plate defining one or more openings for causing the turbulence in the pressurized gas.

According to various exemplary devices described herein, the devices further include a condensation relief valve for releasing moisture from the sanitizing chamber.

According to various exemplary devices described herein, the devices further include a gas relief valve for releasing gas from the sanitizing chamber when the pressure in the sanitizing chamber rises above a predetermined pressure threshold.

According to various exemplary devices described herein, the housing comprises a plurality of upstanding walls defining a first end opening and a second end opening of the sanitizing chamber, a first removable cap for sealing with a light support the first end opening of the upstanding walls, the light support further supporting the light source, and a second removable cap for sealing the second end opening of the upstanding walls.

According to various exemplary devices described herein, the housing comprises a plurality of upstanding walls defining the sanitizing chamber, the upstanding walls being thermally conductive and further comprising at least one heat-sink member for dissipating heat from the sanitizing chamber.

According to various exemplary devices described herein, the plurality of upstanding walls are formed from extrusion.

According to various exemplary devices described herein, the devices further include an ultra-violet sensor for measuring an ultra-violet light level within the sanitizing chamber, an alarm, and a controller for receiving the measured ultra-violet light level and controlling the alarm to emit an alarm indicator when the measured ultra-violet light level falls below a predetermined ultra-violet light threshold.

According to various exemplary devices described herein, the gas outlet is adapted to be connected to a gas-based tool and the water outlet is adapted to be connected to a water-based tool.

According to various exemplary devices described herein, the sanitary chamber is sized based on an expected rate of use of the pressurized gas and a predetermined amount of exposure of pressurized gas to the emitted light.

According to various exemplary devices described herein, the water conduit is sized based on an expected rate of use of the water and a predetermined amount of exposure of water to the emitted light.

The term “microorganism” as used herein refers to microscopic organism including eukaryote and prokaryote organisms, that may exist in water, gas, or any surface, for example in a biofilm. Such organisms may be harmful to humans or animals, such as causing various illnesses in a human or animal when exposed to the organisms or can lead to fouling (e.g. unpleasant taste or odor). Microorganisms can include one or more of bacteria, fungi, algae, archea, protozoa, germs, spores, and viruses.

The term “water-based tool” refers to any tool that is powered by water or that dispenses water. Such tools may be found in the dental field, such as within a dental unit. Such tools may also be found in the medical field, such as tools found in a hospital room, an operating room, or a laboratory.

The term “gas-based tool” refers to any tool that is powered by gas or that dispenses water. Such tools may be found in the dental field, such as within a dental unit. Such tools may also be found in the medical field, such as in tools found in a hospital room, operating room, or a laboratory.

The term “sanitizing” as used herein in relation water and/or gas refers to attenuating and/or killing microorganisms in the water and/or gas, for example, to reduce the number of viable or infectious particles so that they present a lower risk of harm or have less fouling. The attenuating and/or killing may result for example, from inducing damage to the DNA of the microorganisms.

Sanitizing water and/or gas may include attenuating and/or killing the microorganisms in the water and/or gas by at least a factor of 10.

Sanitizing water and/or gas may include attenuating and/or killing the microorganisms in the water and/or gas by at least a factor of 100.

Sanitizing water and/or gas may include attenuating and/or killing the microorganisms in the water and/or gas by at least a factor of 1000.

Sanitizing water and/or gas may include attenuating and/or killing the microorganisms in the water and/or gas by at least a factor of 10000.

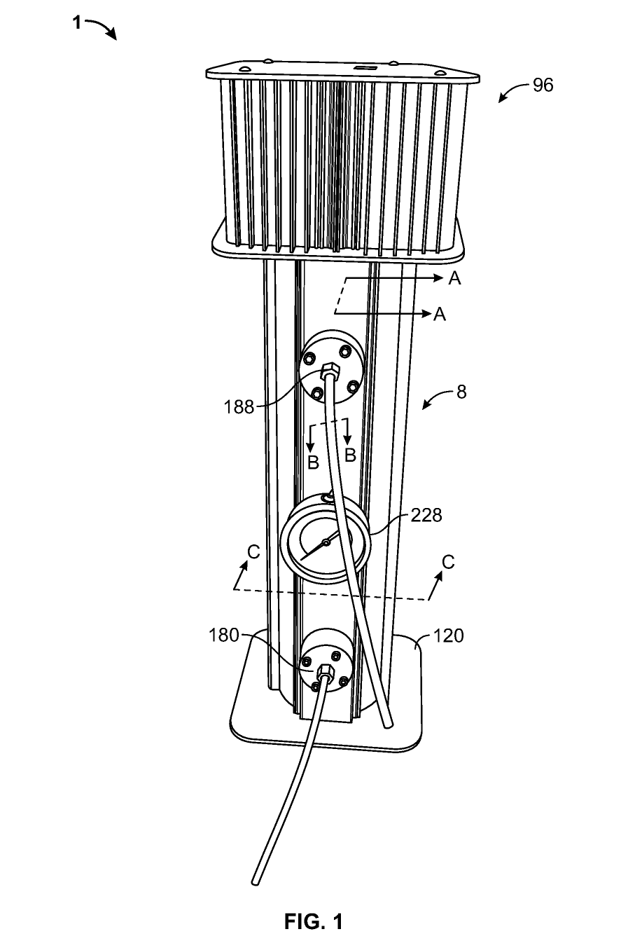

FIG. 1 illustrates a perspective view of a sanitizing device 1 for sanitizing water and pressurized gas.

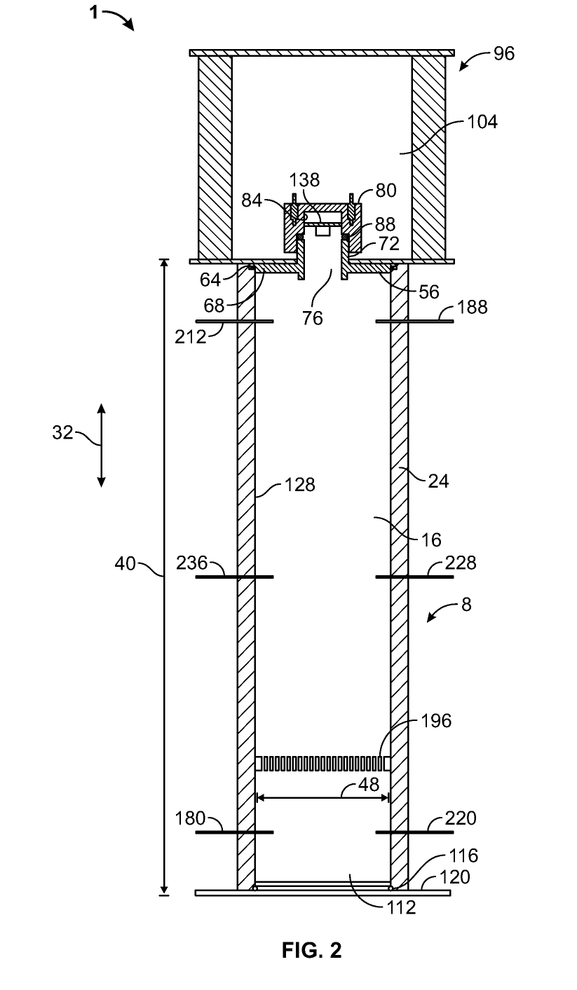

Referring now to FIG. 2, therein illustrated is a cross-sectional view along the line A-A of a sanitizing device 1 according to one exemplary embodiment wherein various sanitizing elements have been removed.

The device 1 includes a first housing 8 defining a sanitizing chamber 16. For example, and as illustrated, the first housing 8 includes upstanding walls 24 extending in an axial direction 32 of the first housing 8. The upstanding walls 24 may extend in the axial direction 32 so that the sanitizing chamber 16 is elongated. For example, the elongated sanitizing chamber may have a length 40 that is substantially greater than a diameter 48 of the chamber. In some exemplary embodiments, the upstanding walls 24 may have a substantially circular inner cross-section, whereby the sanitizing chamber 16 has a generally cylindrical shape. It will be understood that “circular” as used herein includes elliptical shapes.

According to various exemplary embodiments, the upstanding walls 24 define at least a first end opening 56. The first end opening 56 may provide access to a first portion of the sanitizing chamber 16. This may facilitate cleaning, maintenance or replacement of parts within the sanitizing chamber 16. An interior surface of the upstanding walls 24 proximate the first end opening 56 may be internally threaded to cooperate with a first sealing member for fluid-tight sealing of the first end opening 56. The internal threads may be a tapered thread. A first o-ring 64 may be further provided to ensure a fluid-tight seal. For example, the sealing of first end opening 56 with the first sealing member is sufficiently tight to withstand a level of pressure within the sanitizing chamber 16. For example, suitable fasteners, such as high pressure screw caps may be used to fasten the first sealing member to the upstanding walls 24 to ensure tight sealing of the first end opening 56.

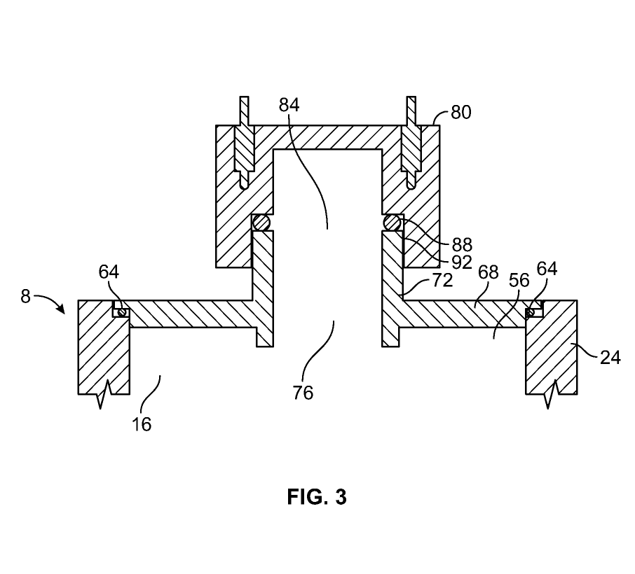

Referring now to FIG. 3, therein illustrated is a cross-sectional view of a region of the first housing 8 in proximity of its first end opening 56. As illustrated, the first sealing member may be a first removable cap 68 adapted to seal the first end opening 56 with first o-ring 64. The first removable cap 68 further includes a tubular portion 72 which extends from a top surface of the first removable cap 68. The tubular portion 72 defines with the first removable cap 68 a center channel 76 providing fluid communication between the sanitizing chamber 16 and an exterior of the first housing 8. The first removable cap 68 may be formed of stainless steel.

A light source (not shown in FIG. 3) may be received within the center channel 76 and project through the center channel 76 to enter the sanitizing chamber 16. A light support 80 may support the light source while sealing a top opening 84 of the center channel 76 of the tubular portion 72. For example, a second o-ring 88 may be positioned between the tubular portion 72 and the light support 80 to further seal the center channel 76. It will be appreciated that the first removable cap 68 and the light support 80 cooperate to seal the first end opening 56 of the sanitizing chamber 16.

Referring back to FIG. 2, the sanitizing device 1 may further include a second housing 96 defining a second chamber 104. The second housing 96 may be supported by or coupled to the first housing 8. The second housing 96 is sized to house the light support 80. The second housing 96 may further house various control components, power supply components and/or ballasts as described elsewhere herein.

The upstanding walls 24 may further define a second end opening 112. The second end opening 112 may provide access to a second portion of the sanitizing chamber 16. This may facilitate cleaning, maintenance or replacement of parts within the sanitizing chamber 16. The interior surface of the upstanding walls 24 proximate the second end opening 112 may also be internally threaded to cooperate with a second sealing member 120 for fluid-tight sealing the second end opening 112. The internal threads at the second end opening 112 may be a tapered thread. A second o-ring 116 may be further provided to ensure a fluid-tight seal. For example, the sealing of second end opening 112 with the second sealing member is sufficiently tight to withstand a level of pressure within the sanitizing chamber 16. For example, suitable fasteners, such as high pressure screw caps may be used to fasten the second sealing member to the upstanding walls 24 to ensure tight sealing of the second end opening 112.

The second sealing member 120 may be a removable cap. According to various exemplary embodiments, the removable cap 120 may have a planar bottom surface and a width greater than an outer diameter of the upstanding walls 24. Accordingly, the second removable cap may also act as a base for supporting the sanitizing device 1. Accordingly, the sanitizing device 1 may be positioned in a vertical upright position, as illustrated. The second removable cap may be formed of stainless steel.

According to various exemplary embodiments, the upstanding walls 24 may be formed of a material that promotes thermal conductance and has high mechanical strength. The material forming the upstanding walls 24 may also be anti-corrosive. In one exemplary embodiment, the upstanding walls 24 are formed of an anodized alloy, such as anodized aluminum alloy. The upstanding walls 24 may be formed of thermally conductive materials, such as various metals, such as stainless steel.

According to various exemplary embodiments where a light source emits UV light, the upstanding walls 24 may be formed of a material that is resistant to long-term exposure to UV rays.

According to various exemplary embodiments, an inner surface 128 of the upstanding walls 24 may be reflective to light. In particular, the inner surface 128 may be reflective to light in the range of wavelengths that is effective for attenuating and/or killing the microorganisms. For example, the inner surface 128 of the upstanding walls 24 may be polished to improve reflectivity to light.

The inner surface 128 may have one or more coatings. A first type of coating may be formed of materials that reduce corrosion of the inner surface 128. A second type of coating may be a non-stick coating, such as Teflon™, to reduce for example accumulation of dust and dirt on the inner surface 128. The one or more coatings may allow passage of at least some sanitizing light in the range of wavelengths effective for sanitizing (i.e. sanitizing light).

The sanitizing chamber 16 is operable to receive therein a light source 136. When received in the sanitizing chamber 16, the light source 136 is operable to emit sanitizing light into the sanitizing chamber 16.

The light source 136 emits a sanitizing light, which has a range of wavelengths that is effective for sanitizing water and/or gas. For example, the light source 136 emits the sanitizing light in the ultraviolet range, such as UV-A, UV-B and/or UV-C, that is effective for sanitizing.

In various exemplary embodiments where the light source 136 emits light source in the UV-C range, a substantial portion of the sanitizing light has wavelengths of approximately 254 nm in length. For example, the sanitizing light has wavelengths in the range of between about 100 nm to about 280 nm. For example, the sanitizing light has wavelengths in the range of about 230 nm to about 280 nm.

The sanitizing light emits light rays that bombard the microorganisms causing damage to the DNA of the microorganisms, thereby attenuating and/or killing the microorganisms. Using adequate sanitizing light may inhibit or reduce the likelihood of any microorganisms from mutating and developing new strains of resistance, as could be the case with use of chemicals for sanitization.

According to various exemplary embodiments, the light source 136 may also emit an amount of ozone, which further assists in sanitizing the water and/or gas. It has been observed that various types of light sources that are characterized as being ozone free may still emit small amounts of ozone that are effective in assisting in sanitizing.

According to various exemplary embodiments, the light source 136 may be a quartz cathode lamp, such as a UVC quartz cathode lamp. However, it will be understood that other types of lamps operable to emit sanitizing light in a range of wavelengths effective for sanitizing water and gas may be used. For example, the light source 136 may include one or more of a UVC-quartz amalgam lamp, UVC-fluorescent lamp and LED lamp.

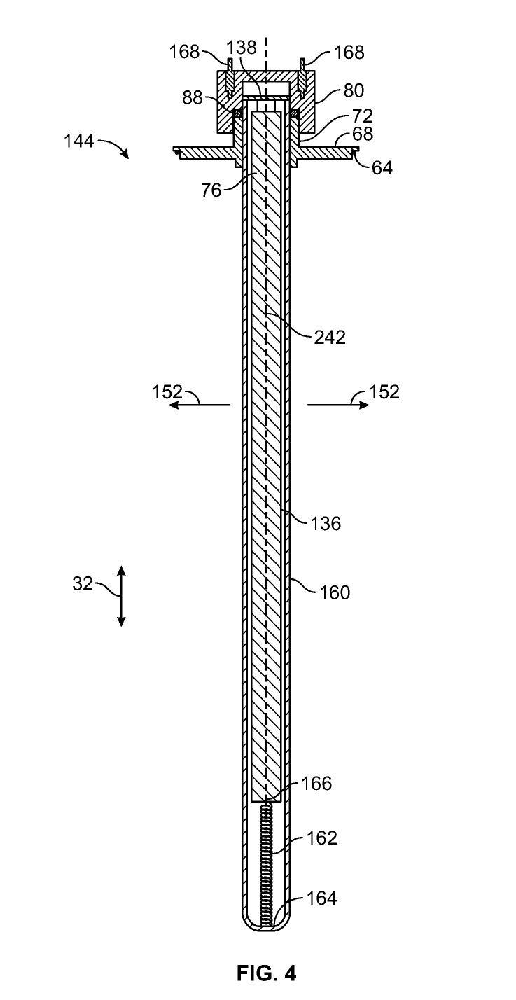

Referring now to FIG. 4, therein illustrated is a cross-sectional view of a lighting mechanism 144 according to one exemplary embodiment. The lighting mechanism 144 includes the light source 136. For example, and as illustrated, the light source 136 is elongated and emits light typically in a radial direction 152. An end portion of the light source 136 may also emit some sanitizing light in a direction in the axial direction 32.

According to various exemplary embodiments, a protective sleeve 160 may be provided to protect the light source 136 from debris and from other components. The protective sleeve 160 may also constrain leakage of material from the light source 136 in case of breakage of the light source 136, such as leakage of mercury where there is a break in a quartz lamp. The protective sleeve 160 allows passage therethrough of sanitizing light.

As illustrated, the light source 136 is coupled to the light support 80. The light support 80 may include electrical leads 168 for receiving a source of electrical power for powering the light source 136. When the light source 136 is coupled to the light support 80 and the light support 80 is further positioned to seal the opening 84 of the center channel 76 of the tubular portion 72 of the removable cap 68, the light source 136 projects through the center channel 76. The protective sleeve 160 may also be supported by the light support 80 and project through the center channel 76.

As illustrated, the light source 136 is located inside of the protective sleeve 160. For example, the protective sleeve 160 may fit snugly within the center channel 76, wherein a portion of the light source 136 extending past the removable cap 68 is completely contoured by the protective sleeve 160.

According to various exemplary embodiments, and as illustrated in FIG. 4, a spring member 162 may be provided within the protective sleeve 160. The spring member 162 engages an inner bottom 164 of the protective sleeve 160 and a distal end 166 of the light source 136 to support the light source 136 within the protective sleeve 160.

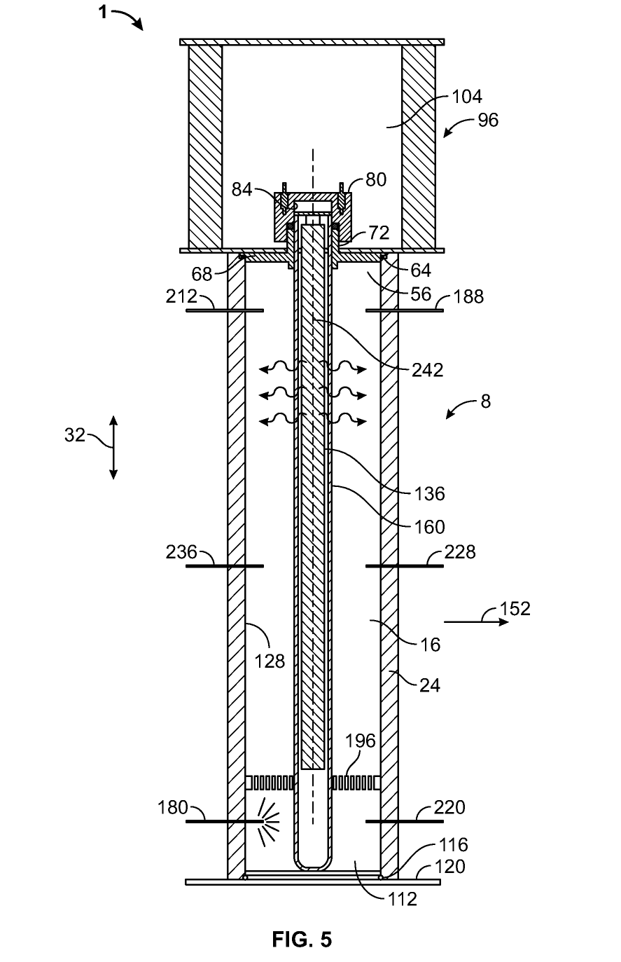

Referring now to FIG. 5, therein illustrated is a cross-sectional view of the exemplary sanitizing device 1 wherein the lighting mechanism 144 has been fitted to the first housing 8. As illustrated, the light source 136 is elongated and extends in the axial direction 32 along a length of the elongated sanitizing chamber 16. For example, the light source 136 extends over at least half of the length of the sanitizing chamber 16.

The protective sleeve 160 extends from the first end opening 56 of the sanitizing chamber 16 to a second end opening 112, whereat it may be supported by a upright members located near the second end opening 112. Accordingly, the protective sleeve 160 may be secured within the sanitizing chamber 16. As illustrated, the protective sleeve 160 forms a complete boundary around the light source 136 to protect it from elements found elsewhere in the sanitizing chamber 16.

The light source 136 may be coupled to a light cap 138 which rests on an upper lip of the protective sleeve 160, thereby supporting the light source 136 within the protective sleeve 160. A spring member may be further provided at the base of the light source 136 within the protective sleeve 160 to provide further supporting of the light source 136. The light cap 138 may include electrical leads for connecting the light source 136 to a power source.

The sanitizing chamber 16 is fluid-tight sealed by the first removable cap 68, light support 80 and second removable cap 120.

Sanitizing light from the light source 136 is emitted in a radial direction 152. Emitted light that is incident on the inner surface 128 of the upstanding walls 24 is reflected by the reflective inner surface 128. The reflected light in combination with the light emitted from the light source 136 contributes to increased sanitizing light within the sanitizing chamber 16.

The sanitizing device 1 includes a gas inlet 180 for receiving pressurized gas. The pressurized gas may be provided through a gas line from a gas storage mechanism, such as a gas tank, or a gas treating mechanism, such as a gas compressor. The pressurized gas has a pressure greater than 1 atm, and may have a pressure up to about 7 atm. The gas inlet 180 may be a push-type inlet.

Due to sealing of the sanitizing chamber 16, pressurized gas received through the gas inlet 180 remains pressurized after entering the sanitizing chamber 16.

The gas inlet 180 may include a pressure control manometer for controlling the pressure of the gas that enters the sanitizing chamber 16 through the gas inlet 180. In various exemplary embodiments, the gas inlet 180 may be controlled to limit the pressure of gas enter the sanitizing chamber 16 through the gas inlet 180.

The gas inlet 180 may be controlled by an operator according to the pressure requirements or pressure operating limits of a downstream gas-based tool that receives gas from the sanitizing device 1. For example in the dental field, gas-based dental tools typically have pressure requirements under 60 psi and the gas inlet 180 may be controlled to provide pressurized gas at between about 10 psi and about 60 psi.

The providing of the gas inlet 180 with a pressure control manometer acts as a pressure safety mechanism for limiting the pressure of gas within the sanitizing chamber 16.

According to various exemplary embodiments, the gas inlet 180 may be automatically controlled based on pressure requirements of a downstream gas-based tool that receives gas from the sanitizing device 1.

The sanitizing device 1 further includes a gas outlet 188 for outputting pressurized gas from the sanitizing chamber 16. The gas outlet 188 may be a push-in type fitting. The gas outlet 188 may be connected to an output gas line, which may be further connected to a gas-based tool, such as a medical tool or a dental tool.

Pressurized gas received at the gas inlet 180 flows through the sanitizing chamber 16 to the gas outlet 188. As the pressurized gas flows through or rests in the sanitizing chamber 16, it is exposed to light emitted from the light source 136 and the emitted light reflecting from the inner surface 128 of the upstanding walls 24. Light incident on the pressurized gas sanitizes the pressurized gas.

The gas inlet 180 may be located in a second end region of the sanitizing chamber 16 proximate the second end opening 112. For example, the second end region corresponds to a bottom end region when the sanitizing device 1 is in an upright position.

The gas outlet 188 may be located in a first end region of the sanitizing chamber 16 proximate the first end opening 56. For example, the first end region corresponds to a top end region when the sanitizing device 1 is in an upright position.

Accordingly, the pressurized gas flows over the length of the sanitizing chamber 16 between the gas inlet 180 and the gas outlet 188, thereby increasing the exposure of the pressurized gas to light emitted from the light source 136.

The pressurized gas may flow freely inside the sanitizing chamber 16. The gas “flowing freely” refers to the pressure gas being able to flow throughout the sanitizing chamber 16 and being only restricted by the presence of other components located within the sanitizing chamber 16. In particular, when the pressurized gas flows freely in the sanitizing chamber 16, its flow is not constrained by some passageway or gas conduit.

According to various exemplary embodiments, the instantaneous flow rate of the gas outlet 188 is chosen based on the maximum instantaneous flow rate of the gas-based tool downstream of the gas outlet 188. For example, the gas outlet 188 is sized so as to be able to supply pressurized gas at the flow rate required by the downstream tool.

The flow rate of the gas inlet 180 may be chosen based on the expected rate of use pressurized gas by the tool downstream of the gas outlet 188. For example, an average flow rate of the gas inlet 188 may be chosen to be greater than average expected rate use of the downstream gas-based tool. Accordingly, the pressurized gas in the sanitizing chamber 16 may be replenished more quickly than it is being used up by the downstream gas-based tool.

According to various exemplary embodiments, the output of the light source 136 and the dimensions of the sanitizing chamber 16 are chosen based on an expected rate of use and a desired and/or minimally required exposure of the pressurized gas to sanitizing light from the light source 136. For example, the sanitary chamber 16 is sized and output of the light source 136 is configured so that a given unit of gas entering the sanitizing chamber 16 via the gas inlet 180 is exposed to an average amount of sanitizing light that is substantially greater than the minimally required amount of exposure to sanitizing light for effective sanitizing. For example, the dimensions of the sanitizing chamber 16 and the output of the light source 136 are chosen so that the amount of sanitizing light emitted into the sanitizing chamber 16 in the time required to deplete a volume of pressurized gas equivalent to the volume of the sanitizing chamber 16 is substantially greater than the minimally required amount of exposure to sanitizing light for a given factor of sanitizing. The time required to deplete the given volume may be calculated based on an expected average rate of use by the downstream gas-based tool. For example, a given unit of gas is exposed so that at least a 2 log rate of micro-organism attenuation and/or killing is attained and preferably at least 4 log rate of micro-organism attenuation and/or killing is attained.

The output of the light source 136 (i.e. irradiance) may be expressed as power per area (e.g. μW/ [symbol] cm [symbol] ̂2). It will be appreciated that this measurement of output of the light source 136 may be based on characteristics of a chosen light source 136 and on a radius of the sanitizing chamber 16. For example, in an elongated sanitizing chamber 16, the output may be measured at the inner surface 128 of the upstanding walls 24 of the sanitizing device 1. It will be appreciated that the output of light may be higher in regions of the sanitizing chamber 16 closer to the light source 136.

In various exemplary embodiments, the light source 136 may have an output of at least about 1000 μW/ [symbol] cm [symbol] ̂2 in the range of wavelengths effective for sanitizing (i.e. sanitizing light) and without taking into account further increased output due to reflection of light within the sanitizing chamber 16.

In various exemplary embodiments, the light source 136 may have an output of at least about 2000 μW [symbol] cm [symbol] ̂2 in the range of wavelengths effective for sanitizing (i.e. sanitizing light)and without taking into account further increased output due to reflection of light within the sanitizing chamber 16.

In various exemplary embodiments, the light source 136 may have an output of at least about 3000 μW/ [symbol] cm [symbol] ̂2 in the range of wavelengths effective for sanitizing (i.e. sanitizing light)and without taking into account further increased output due to reflection of light within the sanitizing chamber 16.

In various exemplary embodiments, the light source 136 may have an output of at least about 4000 μW/ [symbol] cm [symbol] ̂2 in the range of wavelengths effective for sanitizing (i.e. sanitizing light)and without taking into account further increased output due to reflection of light within the sanitizing chamber 16.

The expected amount of time (e.g. seconds) that pressurized gas spends within the sanitizing chamber 16, which corresponds to the amount of time that pressurized gas is exposed to sanitizing light from the light source 136, may be determined based on expected rate of use of gas (e.g litres/minute) and the volume of the sanitizing chamber 16 (e.g. litres). According to various exemplary embodiments where a radius of an elongated sanitizing chamber 16 has already been selected, the volume may be obtained by varying the length of the sanitizing chamber 16.

A total amount of exposure of the pressurized gas to sanitizing light (i.e. radiant exposure expressed, for example, in μJ/ [symbol] cm [symbol] ̂2) may then be calculated from the output of the light source 136 and the expected amount of time that pressurized gas spends within the sanitizing chamber 16.

For example, a light source 136 having an output of at least about 3950 μW/ [symbol] cm [symbol] ̂2 of sanitizing light placed within a sanitizing chamber having a length of about 40.6 cm, a radius of about 4.4 cm and a volume of about 2.5 litres causes pressurized gas flowing through the sanitizing chamber 16 to be exposed to a dosage of at least about 237,000 μJ/ [symbol] cm [symbol] ̂2 where the expected rate of use of pressurized gas is about 2.5 litres/minute.

According to one exemplary embodiment, the output of the light source 136 and the dimensions of the sanitizing chamber 16 are chosen so that pressurized gas flowing through the sanitizing chamber 16 has a total amount of exposure (e.g. dosage) to sanitizing light of at least about 60,000 μJ/ [symbol] cm [symbol] ̂2.

According to one exemplary embodiment, the output of the light source 136 and the dimensions of the sanitizing chamber 16 are chosen so that pressurized gas flowing through the sanitizing chamber 16 has a total amount of exposure (e.g. dosage) to sanitizing light of at least about 120,000 μJ/ [symbol] cm [symbol] ̂2.

According to one exemplary embodiment, the output of the light source 136 and the dimensions of the sanitizing chamber 16 are chosen so that pressurized gas flowing through the sanitizing chamber 16 has a total amount of exposure (e.g. dosage) to sanitizing light of at least about 180,000 μJ/ [symbol] cm [symbol] ̂2.

According to one exemplary embodiment, the output of the light source 136 and the dimensions of the sanitizing chamber 16 are chosen so that pressurized gas flowing through the sanitizing chamber 16 has a total amount of exposure (e.g. dosage) to sanitizing light of at least about 240,000 μJ/ [symbol] cm [symbol] ̂2.

According to various exemplary embodiments, the sanitizing device 1 includes at least one disturbance member positioned within the sanitizing chamber 16. The disturbance member is positioned and sized so as to impede flow of the pressurized gas within the sanitizing chamber 16. Accordingly, contact of pressurized gas with the at least one disturbance member causes turbulence in the flow of pressurized gas. The turbulence in the pressurized gas advantageously promotes mixing of the pressurized gas within the sanitizing chamber 16. Furthermore, the disturbance member may cause the formation of various flow paths through the sanitizing chamber 16 from the gas inlet 180 to the gas outlet 188. The disturbance member may be positioned in proximity for the gas inlet 180 so that flow of pressurized gas received in the sanitizing chamber 16 is disturbed at an early stage.

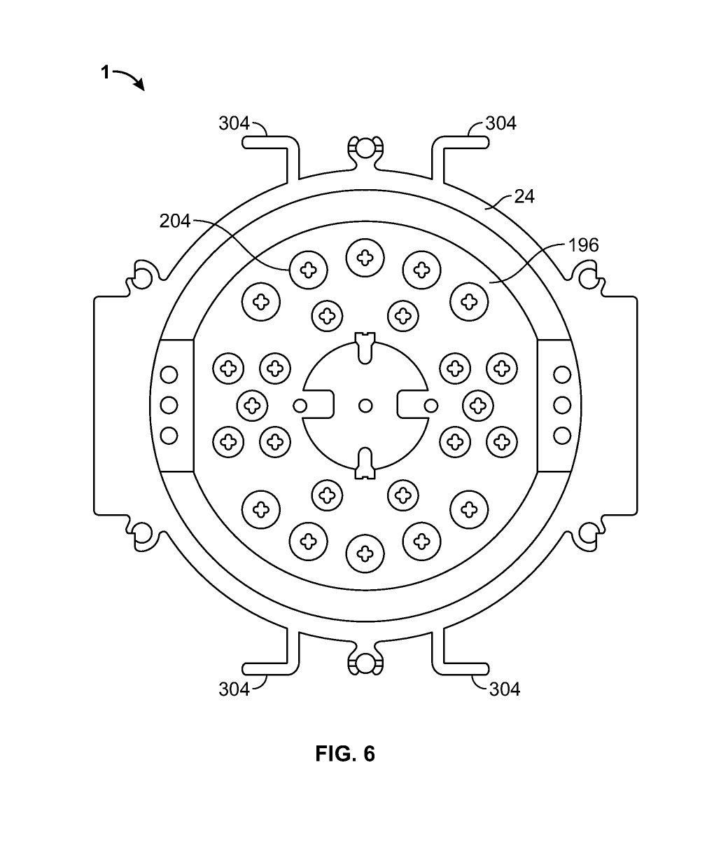

Referring now to FIG. 6, therein illustrated is a cross-sectional view along the line B-B of a sanitizing device 1 according to one exemplary embodiment. According to this exemplary embodiment, the disturbance member may be a baffle plate 196 having at least one discontinuity through which the pressurized gas may be flowed. For example, the baffle plate 196 may have one or more throughholes 204 allowing passage of pressurized gas therethrough. Flow of pressurized gas through the througholes 204 causes turbulence in the pressurized gas. As illustrated in FIG. 5, the baffle plate 196 is oriented transversely to the axial direction 32 and is positioned in proximity of the gas inlet 180.

Referring back to FIG. 5, according to various exemplary embodiments, the sanitizing device 1 may further include a pressure relief valve 212. The pressure relief valve 212 permits release of pressurized gas from the sanitizing chamber 16 when the pressure inside the chamber 16 exceeds a predetermined threshold. The predetermined threshold may correspond to a pressure limit for the downstream gas-based tool using the pressurized gas. Accordingly, the release of gas through the pressure relief valve ensures that the pressure of the pressurized gas provided to the downstream gas-based tool does not exceed the safe limit for that tool. For example, the downstream tool may be a hand piece or drill of a dental unit or medical tool, and the pressure relief valve ensures that the pressure of the pressurized gas supplied thereto is below a safe pressure limit for the hand piece or drill.

Alternatively, the predetermined threshold may correspond to a designed safety limit of the sanitizing device 1.

For example, the pressure relief valve 212 may be set to release gas at a pressure that is higher than the pressure set for the gas inlet 180. Pressure within the sanitizing chamber 16 may be increased due to heating effects within the sanitizing chamber 16. For example, the pressure relief valve 212 may be set to release gas when the pressure reaches about 60 psi, about 70 psi, about 80 psi, about 90 psi, or about 100 psi. In some exemplary embodiments, the pressure relief valve 212 may be set to release gas at even higher pressure levels, such as at about 150 psi or over 200 psi.

According to various exemplary embodiments, the sanitizing device 1 may further include a condensation release valve 220. The condensation release valve 220 permits release of moisture, such as water, from the sanitizing chamber 16. Pressurized gas received via the gas inlet 180 may contain moisture, which may cause buildup of moisture within the sanitizing chamber 16. The moisture may be introduced at the gas storage mechanism or gas treating mechanism upstream of the gas inlet 180. The water condensation relief valve 220 reduces moisture in the sanitizing chamber 16, thereby creating a less permissive environment for the building of microorganisms and biofilms within the sanitizing chamber 16. For example, and as illustrated, the condensation release valve 220 is located in the second end region of the sanitizing chamber 16, which may be the bottom end region of the sanitizing chamber 16. For example, the condensation release valve 220 may be located at the bottom of the housing 8.

In various exemplary embodiments, the water condensation relief valve 220 may be manually operated, whereby a user can intermittently actuate the relief valve 220 to control the moisture inside the sanitizing chamber 16.

Alternatively, the water condensation relief valve 220 may be automatically operated. For example, the water condensation relief valve 220 may be intermittently (e.g. periodically) automatically operated to release moisture. Alternatively, the sanitizing device 1 may further include a moisture sensor and the water condensation relief valve 220 may be actuated to release moisture when the sensed moisture level exceeds a predetermined moisture level.

According to various exemplary embodiments, the sanitizing device 1 may further include a gas pressure gauge 228 for sensing a pressure level within the sanitizing chamber 16. The pressure gauge 228 allows visually monitoring the pressure level within the sanitizing chamber 16. The pressure gauge 228 may also relay measured pressure levels as a data signal to a control module of the sanitizing device 1. For example, and as illustrated, the gas pressure gauge 228 at an intermediate position along the length of the sanitizing chamber 16. Monitoring the measured pressure may be useful for ensuring that pressure within the sanitizing chamber 16 is maintained within appropriate levels and for detecting leaks in the housing 8.

According to various exemplary embodiments, the sanitizing device 1 may further include a photodetector 236 for sensing a light level within the sanitizing chamber 16. The photodetector 236 may be operable to sense light levels within the range of wavelengths of light emitted by the light source 136. For example, the photodetector 235 may sense levels of ultra-violet range light, such as UV-C light (e.g. UV sensor). The sensed light level may be relayed as a data signal to a display and/or a control module of the sanitizing device 1.

One or more of the gas inlet 180, gas outlet 188, pressure relief valve 212, water condensation relief valve 220, gas pressure gauge 228 and photodetector 236 may be installed by machining a bore within the upstanding walls 24 and fitting the corresponding element into the bore. For example, the bore may have a tapered thread, such as a National Pipe Thread Taper (NPT), wherein fitting the corresponding element also provides a fluid-tight seal of the bore.

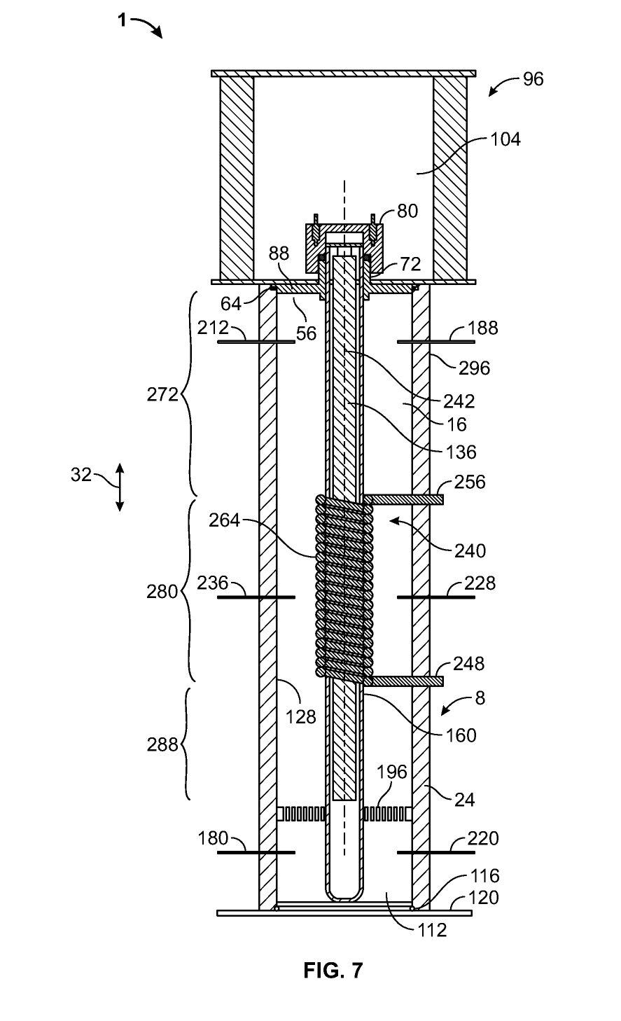

Referring now to FIG. 7, therein illustrated is a cross-sectional view of the sanitizing device having a water conduit 240. The water conduit 240 has a water inlet 248, a water outlet 256, and an intermediate portion 264. Water from a water source is received at the water inlet 248 and further flow through a channel defined by the water conduit 240 to reach the water outlet 256. The water outlet 256 may be connected to an output water line, which may be further connected to a water-based tool, such as a medical tool or a dental tool.

The intermediate portion 264 of the water conduit 240 extends through the sanitizing chamber 16. For example, and as illustrated, the intermediate portion 264 of the water conduit 240 coils around the light source 136. For example, the water conduit 240 may be coiled helically around the light source 136.

For example, and as illustrated, the coiling of the water conduit 240 may be frictionally supported by the protective sleeve 160. However, it will be understood that in other exemplary embodiments, the coiling of the water conduit 240 is radially spaced apart from the protective sleeve 160. The spacing of the water conduit 240 from the protective sleeve 160 may promote disturbance of flow of pressurized gas, as described elsewhere herein.

For example, a the water conduit 240 may be designed to frictionally engage the protective sleeve 160, but through flexing of the water conduit 240 the water conduit 240 loosens around the protective sleeve 160 and becomes spaced apart from it.

At least a portion of the water conduit 240 extending through the sanitizing chamber 16 allows passage of at least some light through it. In particular, the portion allows passage of at least some sanitizing light in the range of wavelengths of light emitted from the light source 136. For example, the portion of the water conduit 240 allows passage of at least about 60%, about 70%, about 80% or about 90% of the sanitizing light. The portion of the water conduit 240 may be formed of a flexible, at least partially transparent material, such as Teflon™, such as fluorinated ethylene propylene (FEP). The FEP may be FDA and/or NSF approved for medical use.

Light emitted from the light source 136 is incident on the water conduit 240 extending through the sanitizing chamber 16. Since at least a portion of the water conduit 240 is at least partially transparent, light incident on the water conduit 240 is further incident on the water flowing through the water conduit 240. Accordingly, water being channeled from the water inlet 248 to the water outlet 256 is exposed to the light emitted from the light source 136 and the emitted light sanitizes the water.

Unlike the pressurized gas that flows freely in the sanitizing chamber 16, the flow of water through the sanitizing chamber 16 is constrained to the channel defined by the water conduit 240. Accordingly, although both the pressurized gas and the water flow through the sanitizing chamber 16, the pressurized gas is physically separated from the water by the walls of the water conduit 240. That is, the pressurized gas and the water are not in contact within the sanitizing device 1. Furthermore, since the pressurized gas flows from a gas storage mechanism or gas treating mechanism to the sanitizing device 1 and further onto a tool using the gas and since the water flows from a water source to the sanitizing device 1 and further onto a tool using the water, the pressurized gas and the water to be sanitized are not generally in contact at any point before being ultimately used or dispensed from their respective tools.

According to various exemplary embodiments, the output of the light source 136 and the dimensions of the water conduit 240 are chosen based on an expected rate of use and a desired and/or minimally required exposure of the water to sanitizing light from the light source 136. For example, the water conduit 240 and output of the light source 136 is configured so that a given unit of water flowing through the sanitizing chamber 16 is exposed to an average amount of sanitizing light that is substantially greater than the minimally required amount of exposure to sanitizing light for effective sanitizing. For example, the size of the water conduit 240 and the output of the light source 136 are chosen so that the amount of sanitizing light emitted into the sanitizing chamber 16 in the time required to deplete a volume of water equivalent to the volume of the water conduit 240 is substantially greater than the minimally required amount of exposure to sanitizing light for a given factor of sanitizing. The time required to deplete the given volume may be calculated based on an expected average rate of use by the downstream water-based tool. For example, a given unit of water is exposed so that at least a 4 log rate of micro-organism reduction is attained.

The output of the light source 136 (i.e. irradiance) may be expressed as power per area (e.g. μW/ [symbol] cm [symbol] ̂2). For example, in an elongated sanitizing chamber 16, the output may be measured at the inner surface 128 of the upstanding walls 24 of the sanitizing device 1. In various exemplary embodiments, the light source 136 may have an output of at least about 1000 μW/ [symbol] cm [symbol] ̂2, 2000 μW/ [symbol] cm [symbol] ̂2, at least about 3000 μW/ [symbol] cm [symbol] ̂2, or at least about 4000 μW/ [symbol] cm [symbol] ̂2 in the range of wavelengths effective for sanitizing (i.e. sanitizing light) and without taking into account further increased output due to reflection of light within the sanitizing chamber 16. It will be appreciated that the output of light may be higher for water conduit 240 as it is located in in regions of the sanitizing chamber 16 closer to the light source 136.

The expected amount of time (e.g. seconds) that water spends within the sanitizing chamber 16, which corresponds to the amount of time that water is exposed to sanitizing light from the light source 136, may be determined based on expected rate of use of water (e.g in litres/minute) and the volume of the water conduit (e.g. in litres).

A total amount of exposure of the pressurized water to sanitizing light (i.e. radiant exposure expressed, for example, in μJ/ [symbol] cm [symbol] ̂2) may then be calculated from the output of the light source 136 and the expected amount of time that water spends within the sanitizing chamber 16.

For example, a light source 136 having an output of at least about 3950 μW/ [symbol] cm [symbol] ̂2 of sanitizing light placed within a water conduit 240 having a portion located in the sanitizing chamber 16 having a length of approximately 600 cm, an inner diameter of 5 mm and a volume of about 0.46 litres causes water flowing through the sanitizing chamber 16 to be exposed to a dosage of at least about 1,090,000 μJ/ [symbol] cm [symbol] ̂2 where the expected rate of use of water is about 0.1 litre/minute.

According to one exemplary embodiment, the output of the light source 136 and the dimensions of the sanitizing chamber 16 are chosen so that water flowing through the sanitizing chamber 16 has a total amount of exposure (e.g. dosage) to sanitizing light of at least about 250,000 μJ/ [symbol] cm [symbol] ̂2.

According to one exemplary embodiment, the output of the light source 136 and the dimensions of the sanitizing chamber 16 are chosen so that water flowing through the sanitizing chamber 16 has a total amount of exposure (e.g. dosage) to sanitizing light of at least about 500,000 μJ/ [symbol] cm [symbol] ̂2.

According to one exemplary embodiment, the output of the light source 136 and the dimensions of the sanitizing chamber 16 are chosen so that water flowing through the sanitizing chamber 16 has a total amount of exposure (e.g. dosage) to sanitizing light of at least about 750,000 μJ/ [symbol] cm [symbol] ̂2.

According to one exemplary embodiment, the output of the light source 136 and the dimensions of the sanitizing chamber 16 are chosen so that water flowing through the sanitizing chamber 16 has a total amount of exposure (e.g. dosage) to sanitizing light of at least about 1,000,000 μJ/ [symbol] cm [symbol] ̂2.

It will be appreciated that these exposure amounts exceed the 186,000 μJ/ [symbol] cm [symbol] ̂2 threshold for destruction of viruses and also exceeds the NSF International's recommended dosage of 40,000 μJ/ [symbol] cm [symbol] ̂2 for water disinfection.

In various exemplary embodiments, it will be appreciated that sanitizing light emitted from the light source 136 into the sanitizing chamber 16 is incident on both the water flowing through the water conduit 240 and the gas flowing through the sanitizing chamber 16 between the gas inlet 180 and the gas outlet 188. For example, a single light source 136 exposes light to both the water and the pressurized gas. Accordingly, the light source 136 performs a dual-sanitizing action in sanitizing both the water and the pressurized gas, which may result in more efficient use of the light source 136.

In some exemplary embodiments, some of the sanitizing light emitted from the light source 136 may first contact a first outer surface of the water conduit 240 and the water flowing through it. Since the water conduit 240 is at least partially transparent, the light may continue out of a second outer surface of the water conduit 240 to be incident on the pressurized gas inside the sanitizing chamber 16. Exposure of water and pressurized gas to sanitizing light from the light source 136 may be further increased due to reflection of light off of the reflective inner surface 128 of the upstanding walls.

It will be understood that tight coiling of the water conduit 240 illustrated in FIG. 7 is shown for exemplary purposes. Tight coiling of the water conduit 240 refers to adjacent revolutions of the coil of the water conduit 240 contacting one another. In other examples, the water conduit 240 may be loosely coiled around the light source 136, wherein at least some of the adjacent revolutions of the coiled are spaced from one another.

The water conduit 240 in the sanitizing chamber 16 may impede the flow of pressurized gas through the sanitizing chamber 16, thereby further causing turbulence of the pressurized gas in the sanitizing chamber 16. In various examples where the water conduit 240 is loosely coiled around the light source 136, causing of turbulence may be further promoted due to pressurized gas flowing through the gaps between adjacent revolutions of the coiled water conduit 240.

According to various exemplary embodiments, the water conduit 240 is coiled about only a portion of the length of the light source 136 and a remainder of the length of the light source 136 is free of the water conduit 240. The water conduit 240 being coiled about a length of the light source 136 refers to the water conduit 240 occupying a space within a plane perpendicular to the axis 242 of the light source 136 at a given lengthwise position of the light source 136. A length of the light source 136 being free of the water conduit 240 refers to the planes perpendicular to the axis of the light source 136 along that length not being occupied by the water conduit 240.

For example, for the exemplary sanitizing device 1 illustrated in FIG. 7, a first lengthwise portion 272 of the light source 136 is free of the coiled water conduit 240, a second lengthwise portion 280 of the light source 136 is surrounded by the coiled water conduit 240, and a third lengthwise portion 288 of the light source 136 is also free of the coiled water conduit 240.

According to various exemplary embodiments, at least a quarter of the length of the light source 136 is free of the coiled water conduit 240.

According to various exemplary embodiments, up to about a half of the length of the light source 136 is free of the coiled water conduit 240.

According to various exemplary embodiments, the upstanding walls 24 may promote dissipation of heat from the sanitizing chamber 16. Emission of light from the light source 136 may cause heating of the sanitizing chamber 16 and various elements located therein. Excessive heating should be avoided due to various reasons. For example, high heat may reduce efficiency of the light source 136. High heat may also cause heating of the water in the water conduit 240, which may be undesirable where the water is to be subsequently administered to human user. High heat may also cause an increase in pressure in the pressurized gas.

The upstanding walls 24 promote heat dissipation due their being formed of materials having high thermal conductance. Accordingly, heat in the sanitizing chamber 16 causes heating of the inner surface 128 of the upstanding walls 24, which is conducted to the outer surface 296 of the upstanding walls 24 and dissipated to the environment surrounding the sanitizing device 1.

According to various exemplary embodiments, a combination of the light source 136 and the heat dissipating upstanding walls 24 may be chosen together so that the temperature within the sanitizing chamber 16 is maintained below a given temperature limit. For example, the temperature within the sanitizing chamber 16 is maintained below approximately 42° C. when the sanitizing device 1 is placed in an environment having an ambient temperature of 21° C. The 42° C. temperature corresponds to a temperature where a mercury lamp as a light source 136 begins to lose performance.

For example, the temperature within the sanitizer chamber 16 is maintained below approximately 37° C. when the sanitizing device 1 is placed in an environment having an ambient temperature of 21° C. The 37° C. temperature corresponds to body temperature for humans and may be more comfortable for humans where the water and/or air is to be dispensed for treating humans.

According to various exemplary embodiments, the outer surface 296 may further include one or more heat-sink members 304 for promoting heat dissipation. Referring back to FIG. 6, the upstanding walls 24 include four heat-sink members 304 extending outwardly from the outer surface 296 of the upstanding walls 24. As illustrated, the heat-sink members 304 may be thin fin-like members having large exposed surfaces for dissipation of heat. It will be understood that any number of heat-sink members 304 of the same or different shapes may be provided on the outer surface 296 of the upstanding walls 24.

In one exemplary embodiment, the heat-sink members 304 extend along the length of the upstanding walls 24. The cross-sectional shape of the upstanding walls 24 along their entire length may be the same so that the upstanding walls 24 may be formed from an extrusion process. The upstanding walls 24 may further include fastening elements for receiving fasteners positioned in an axial direction. The fasteners may be used to secure the first removable cap 68 and/or the second removable cap 120 to ends of the upstanding walls 24 when sealing ends of the sanitizing chamber 16.

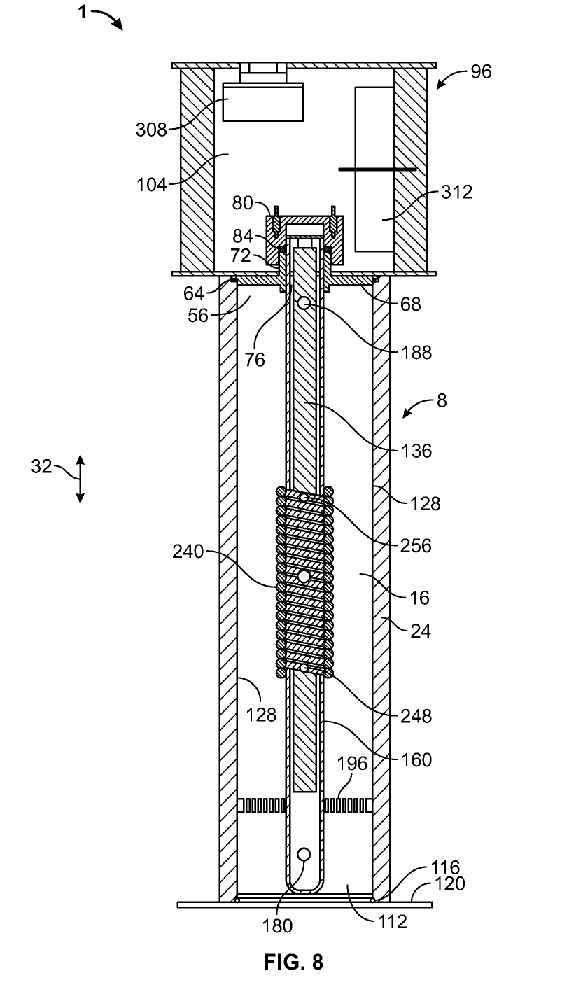

Referring now to FIG. 8, therein illustrated a cross-sectional view along the line C-C of a sanitizing device 1 according to various exemplary embodiments. In the illustrated example, a display device 308 is positioned within the second housing 96. The display device 308 is positioned so that the visible screen of the display device 308 is oriented outwardly. The display device 308 may be display various measured conditions within the sanitizing device 1, such as pressure level in the sanitizing chamber 16, UV light level, and remaining lamp life. The display device 308 may further display the presence of one or more pre-alarms or full alarms as disclosed elsewhere herein.

A ballast 312 may be further positioned in the second housing 96. The ballast 312 houses various electrical components of the sanitizing device 1, such a power supply and controller, as described elsewhere herein.

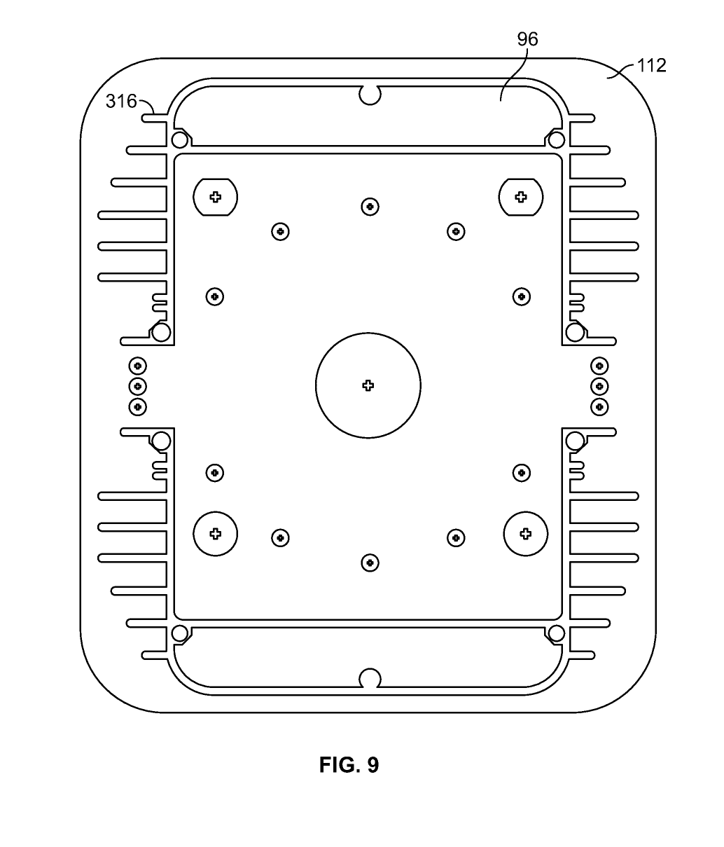

Referring now to FIG. 9, therein illustrated is a plan view of the sanitizing device 1. As illustrated, the second housing 96 includes a plurality of heat-sink members 316 for dissipating heat generated from electrical devices located within the second chamber 104.

A base of the sanitizing device 1, which may be the second removable cap 120 is also visible in FIG. 9.

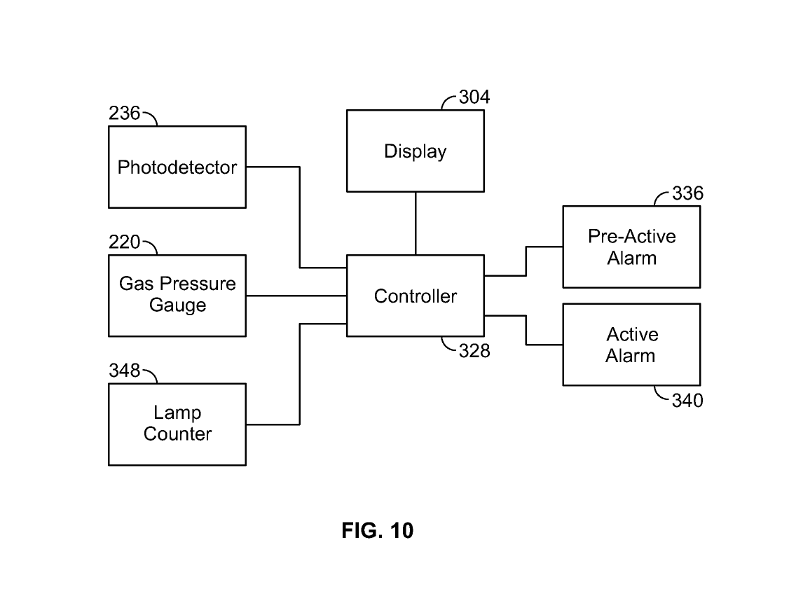

Referring now to FIG. 10, therein illustrated is a block diagram of the operation components of a monitoring system 320 for monitoring components of the sanitizing device 1. The monitoring system 320 may be integrated as part of the sanitizing device 1.

The monitoring system 320 includes a controller 328. The controller 328 described herein may be implemented in hardware or software, or a combination of both. It may be implemented on a programmable processing device, such as a microprocessor or microcontroller, Central Processing Unit (CPU), Digital Signal Processor (DSP), Field Programmable Gate Array (FPGA), general purpose processor, and the like. In some embodiments, the programmable processing device can be coupled to program memory, which stores instructions used to program the programmable processing device to execute the controller. The program memory can include non-transitory storage media, both volatile and non-volatile, including but not limited to, random access memory (RAM), dynamic random access memory (DRAM), static random access memory (SRAM), read-only memory (ROM), programmable read-only memory (PROM), erasable programmable read-only memory (EPROM), electrically erasable programmable read-only memory (EEPROM), flash memory, magnetic media, and optical media.

The monitoring system 320 may include the photodetector 236 (e.g. UV sensor) of the sanitizing device 1 and may receive therefrom one or more data signals that include light level (e.g. UV level) measurements made by the photodetector 236. The measured light level may be displayed on the display device 308.

According to various exemplary embodiments, the controller 328 receives the data signals indicating light level measurements from the photodetector 236. The controller 328 may further compare one or more of the light level measurements against a first light level threshold. The measured light level may be a light level for a wide range of wavelengths. The measured light level may be a light level for a range of wavelengths that includes the sanitizing light. The first light level threshold may correspond to a level of sanitizing light within the sanitizing chamber 16 at which the sanitizing light in the sanitizing chamber 16 is effective for sanitizing fluid—the pressurized gas and/or the water, but is approaching a level where the light becomes ineffective.

Where a measured light level falls below the first light level threshold, the controller 328 may control one or more components to emit a pre-alarm. For example, the controller 328 may control the display device 308 to display a warning message indicating that the light level is becoming significantly low. The controller 328 may further control a pre-alarm device 336 to emit a pre-alarm. The pre-alarm device 336 may be a light indicator or an audio device operable to emit an audible pre-alarm.

The controller 328 may further compare one or more of the light level measurements against a second light level threshold. The second light level threshold may correspond to a level of light within the sanitizing chamber 16 that is no longer effective for sanitizing the pressurized gas and/or the water.

When a measured light level falls below the second light level threshold, the controller 328 may control one or more components to emit a full alarm. For example, the controller 328 may control the display device 308 to display a message informing of a critical status. The controller 328 may further control an alarm device 340 to emit a full alarm. The alarm device 340 may be a light indicator or an audio device operable to emit an audible full alarm. The full alarm differs from the pre-alarm to allow a user to distinguish between the pre-alarm and the full alarm.

The monitoring system 320 may include a gas pressure gauge 228 of the sanitizing device 1 and may receive therefrom one or more data signals that include pressure level measurements made by the gas pressure gauge 228.

The controller 328 may further compare one or more of the pressure level measurements against a first low pressure threshold. The first low pressure threshold may correspond to a pressure level within the sanitizing chamber 16 remains within a usable pressure range but is approaching a level where the pressure level becomes unusable. A usable pressure range refers to a range wherein pressurized gas outputted to a downstream gas-based tool via the gas outlet 188 has sufficient pressure to meet the purpose of the gas-based tool.

Where the measured pressure level falls below the first low pressure level threshold, the controller 328 may control one or more components to emit a pre-alarm. For example, the controller 328 may control the display device 308 to display a warning message indicating that the pressure level is becoming significantly low. The controller 328 may further control a pre-alarm device 336 to emit a pre-alarm. The pre-alarm device 336 may be a light indicator or an audio device operable to emit an audible pre-alarm.

The controller 328 may further compare one or more of the pressure level measurements against a second low pressure level threshold. The second low pressure level threshold may correspond to a pressure level within the sanitizing chamber 16 wherein gas outputted via the gas outlet 188 is no longer sufficiently pressurized for use by the downstream gas-based tool.

When a measured light level falls below the second low pressure level threshold, the controller 328 may control one or more components to emit a full alarm. For example, the controller 328 may control the display device 308 to display a message informing of a critical status. The controller 328 may further control the alarm device 340 to emit a full alarm.

Low pressure levels may be caused by a leak in the sanitizing device 1 or upstream of the sanitizing device 1. Low pressure levels may also be caused by a failure of a gas treatment mechanism, such as the air compressor.

The controller 328 may further compare one or more of the pressure level measurements against a first high pressure threshold. The first high pressure threshold may correspond to a pressure level within the sanitizing chamber 16 that remains within a usable pressure range but is approaching a level where the pressure level becomes too high. A pressure level may be too high in that it exceeds the range of pressure for operating the downstream gas-based too. A pressure may also be too high in that it exceeds safe pressure levels of the housing 8 of the sanitizing device 1.

Where the measured pressure level rises above the first high pressure level threshold, the controller 328 may control one or more components to emit a pre-alarm. For example, the controller 328 may control the display device 308 to display a warning message indicating that the pressure level is becoming significantly high. The controller 328 may further control a pre-alarm device 336 to emit a pre-alarm. The pre-alarm device 336 may be a light indicator or an audio device operable to emit an audible pre-alarm.

The controller 328 may further compare one or more of the pressure level measurements against a second high pressure level threshold. The second high pressure level threshold may correspond to a pressure level within the sanitizing chamber 16 wherein gas outputted via the gas outlet 188 is too high to be used by the downstream gas-based tool or has exceeded safe pressure levels of the first housing 8 of the sanitizing device 1.

When a measured light level rises above the second high pressure level threshold, the controller 328 may control one or more components to emit a full alarm. For example, the controller 328 may control the display device 308 to display a message informing of a critical status. The controller 328 may further control the alarm device 340 to emit a full alarm.

A high pressure may be due to a blockage in the sanitizing device 1 or upstream or downstream of the sanitizing device 1. A high pressure may also be due to over pressurization when operating one or more devices in the sanitizing device 1 or upstream or downstream of the sanitizing device 1.

The monitoring system may further include a lamp counter 348. The lamp counter 348 tracks the time a current light source 136 has been in operation. The current count of the lamp counter 348 may be received at the controller 328.

The controller 328 may compare one or more received current counts against a first count threshold. The first count threshold may correspond to an operation age of the light source 136 wherein the light source is still effective but is approaching an operation age where the light source will become ineffective.

Where the received current count exceeds the first count threshold, the controller 328 may control one or more components to emit a pre-alarm. For example, the controller 328 may control the display device 308 to display a warning message indicating that the age of the current light source 136 is nearing its end. The controller 328 may further control a pre-alarm device 336 to emit a pre-alarm. The pre-alarm device 336 may be a light indicator or an audio device operable to emit an audible pre-alarm.

The controller 328 may further compare one or more received counts against a second count threshold. The second count threshold may correspond to an operation age of the light source 136 wherein the light source should no longer be able to emit light effective for sanitizing gas or water.

When the received current count exceeds the second count threshold, the controller 328 may control one or more components to emit a full alarm. For example, the controller 328 may control the display device 308 to display a message informing of a critical status. The controller 328 may further control the alarm device 340 to emit a full alarm.

The count may be reset whenever a lamp of the light source is replaced. The first count threshold or second count threshold may also be modified based on the type and/or model of the newly installed light source.

According to a method for sanitizing pressurized gas and/or water with the sanitizing device 1, at least one of pressurized gas and water is received within the sanitizing chamber 16. The pressurized gas and/or water is further exposed to light in a range of wavelengths that is effective for sanitizing the pressurized gas and/or water. At least one condition within the sanitizing chamber 16 and/or a condition of the sanitizing device 1 measure. For example, the at least one status may be intermittently monitored. For example, the at least one condition may be one or more of pressure level in the sanitizing chamber 16, light level (e.g. UV level) in the sanitizing chamber and operation age of the light source emitting the light.

The method further includes comparing the at least one condition with at least one predetermined threshold corresponding to that condition. Various predetermined thresholds have been described herein with reference to the controller 328. Where a given measurement goes beyond a given threshold, the method further includes emitting an alarm. As described herein with reference to the controller 328, a pre-alarm may be emitted when the measurement goes beyond a first threshold. A full alarm can then be emitted when the measurement goes beyond a second threshold.

Various exemplary embodiments described herein may be used to sanitize fluids from a source of pressurized gas and/or a source of water prior to use by a water-based tool and/or gas-based tool downstream of the gas outlet 188 or water outlet 256. The sanitizing devices and methods described herein may be used in the medical field or dental field.

In such fields, sanitizing gas and/or water may be important where the gas and/or water is applied to immune system suppressed patients, such as young children and elderly persons. For example, the sanitizing of gas and/or water may further decrease the risk of such patients being affected by microorganisms in the gas and/or water.

In such fields, sanitizing may also be important for operations that expose a sensitive part of a patient to the gas and/or water. For example, the sanitizing may be important when used in open wound procedures and drilling procedures, such a drilling into bone.

In a method of using the sanitizing device 1 described herein, the sanitizing device 1 is located in proximity of the gas-based tool and/or water-based tool that will be using the pressurized gas and/or water. The gas outlet 188 of the sanitizing device 1 may be directly connected to the gas-based tool. Similarly, the water outlet 256 of the sanitizing device may be directly connected to the water-based tool. Directly connected to a water-based tool refers to the outlet being connected tool whereby the length of the gas or water piping providing the connection is minimized. For example, being directly connected includes the water-based tool or gas-based tool being connected to the water outlet 256 or gas outlet 256 free of any intermediate devices that may further treat the water or pressurized gas. For example, the sanitizing device 1 may represent the final stage to the flow of pressurized gas and/or water before it is used by the gas-based tool and/or water-based tool.

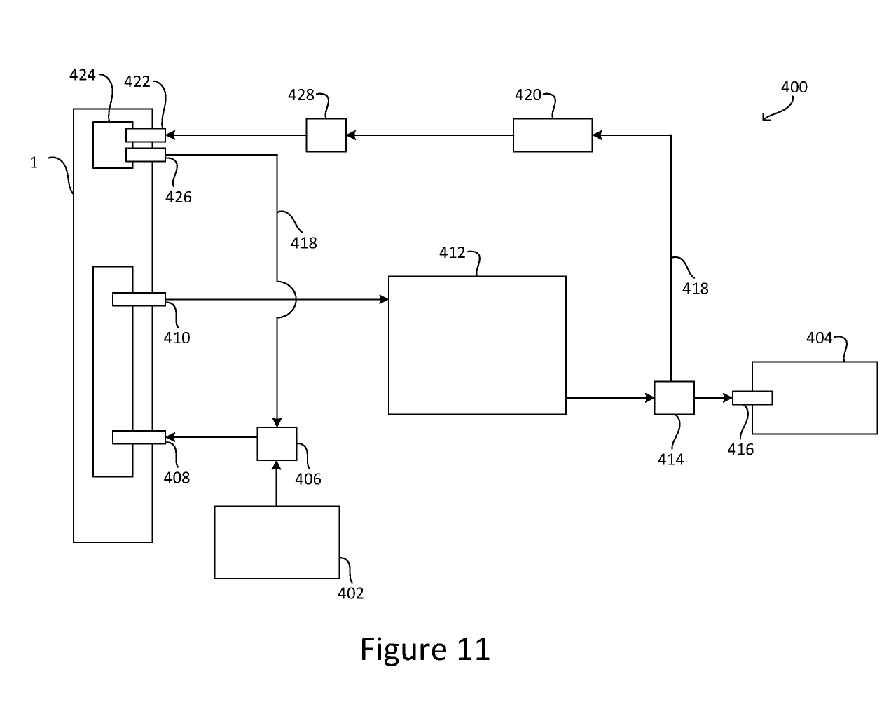

Alternatively, the water outlet 256 of the sanitizing device may be connected to a re-circulating system, for example as described with reference to FIG. 11.

Advantageously, by positioning the sanitizing device 1 at the final stage, the sanitizing device 1 is operative to sanitize the pressurized gas and/or water upstream of the sanitizing device 1. Furthermore, potential sources for any build-up of microorganism downstream of the sanitizing device 1 are minimized.

According to various exemplary embodiments where the water conduit 240 extends through the sanitizing chamber 16 and where pressurized gas flows through the sanitizing chamber 16, the space required for sanitizing the pressurized gas and the water is reduced because sanitizing of two types of fluids may be carried out in a single sanitizing chamber 16.

The space saving may become important in various applications. For example, in a dental room, hospital room, or operating room, space may be limited due to presence of other equipment. The smaller space occupied by the sanitizing device 1 may allow it to be placed in the dental room, hospital room, or operating room, whereby the gas outlet 188 and the water outlet 256 may be connected to their respective tools for dispensing pressurized gas and water.

Referring now to FIG. 11, illustrated therein is a recirculation system 400, in accordance with an embodiment. The recirculation system 400 may be retrofitted into existing units and may also be used in the design of a new unit. The recirculation system 400 connects between a water source 402 and a tool 404.

The water source 402 may be a water supply and ultimately connect to a water tank or municipal water supply. The tool 404 may be a medical or dental surgical hand tool that uses both water and gas (for example as dentist's hand tool). The recirculation system 400 includes a recirculation return tube 418 for recirculating water from the sterilization device 1. The recirculation system 400 is a closed circuit of water flowing through the tubing to the tool 404. The water is recirculated through the sterilization device 1 where pathogens are removed that may be contained in the tubing of the system 400, while some of the water is able to be used by the tool 404 (e.g., hand drills and hand tools).

The recirculation return tube 418 cycles the split water back to where the water source 402 enters the sterilization device 100. The recirculation return tube 418 may be a plastic tube having an inner diameter from 1 mm to 6 mm. In a particular embodiment, the recirculation return tube 418 is 1.5 mm in inner diameter.

The system 400 includes a water source connection 406 that connects recirculated water with the water source 402 before the water enters the sterilization device 1. The water source connection 406 fluidly connects the water source 402 and the recirculation return tube 418 to a water inlet 408 of the sterilization device.

The water source 402 water and recirculating water enter the UV reactor of the sanitizing system 1 via the water inlet 408 (e.g., water inlet 248 of FIG. 7). The water is treated by the sanitizing system 1 and the treated water exits the sanitizing device 1 at a water outlet 410 (e.g., water outlet 256 of FIG. 7).

The treated water exiting the water outlet 410 is fed into the water control circuit 412. The water control circuit 412 may be a dental or medical unit water control that uses gas to power the tool 404. The water exits the water control circuit 412 and enters a tool connection 414.

Some of the water from the tool connection 414 will pass through a tool joint 416 to the tool 404, while some of the water will enter the recirculation return tube 418. The tool connection 414 fluidly connects the water control circuit to the tool 404 and the recirculation return tube 418. The tool connection 414 may have a small bypass prior to the tool connection 414 that splits the water flow by 30% to 50% of the total water flow.

The water source connection 406 and tool connection 414 may be a T valve, a Y valve, a bypass valve, or the like. The water source and tool connections 406, 414 may be quick connecting, such that a user can install the device to an existing system. The water source and tool connections 406, 414 may be of molded plastic, metal, or may be of machined metal. The water source and tool connections 406, 414 hook up to existing tubes and may not to take much space inside the bundle of tubes as there may already be a plurality of tubes connected to the tool 404. For example, a dental hand piece/drill inside a plastic jacket may have from 4 to 6 existing tubes leading to it.

The recirculation system 400 may include a recirculation pump 424 for pumping water in the recirculation return 418 causing the water to flow and recirculate. The water in the recirculation return tube 418 is pumped into a water pump inlet 422, through the water pump 424, and out of the water pump outlet 426. The water pump 424 may be integrally formed within the housing of the sterilizing device 1. Water exiting the water pump outlet 426 connects back to the water source connection 406.

The pump 424 may be running continuously, when the recirculation system 400 is turned on. The pump 424 recirculates the water from the tool connection 414 and may provide 24 hours a day of water flowing through the recirculation system 400. Further, the recirculation system 400 may destroy pathogens shed by the biofilm throughout the day and the night. While, the American Dental Association (ADA) has recommended having at least 2 to 8 minutes/day of running water pass through the dental equipment circuit, the recirculation system 400 may continuously prevent water from becoming or staying stagnant. Where the water control circuit 412 is new, the recirculation system 400 running 24 hrs/day may inhibit biofilm adhesion to the inner walls of the tubes due to the constant flow of the treated water in the tubes. The pump 424 running constantly can be replaced as needed (for example, every few years or earlier). In an embodiment a timing system turns the pump 424 on and off at a duty of less than 50% for a longer life if desired by the manufacturer for a longer pump life span.

In an embodiment, the recirculation return tube 418 may have a one way check valve 420 in the recirculation return tube 418 to prevent water from back flowing and bypassing the treatment at sanitizing device 1.

In an embodiment, the return tube 418 includes a flow sensor 428 in the recirculation return tube 418. The flow sensor 428 determines if water is flowing in the recirculation return tube 418. When the determined flow is below a predetermined level, the recirculation system 400 will trigger an alarm. For example, the recirculation includes an alarm buzzer (e.g. of control system of FIG. 10) where water is not flowing in the recirculation return tube 418. In some cases, the flow sensor 428 will trigger the alarm if the water is not flowing for more than a predetermined amount of time. A user, when hearing the alarm, is then able to check the recirculation system 400, to have the flow resume.

The recirculation system 400 may include a series flow constrictor (not shown) that constricts the water flow level amount to assure the adequate sterilization (UV dosage) even at a maximum flow level. For example the water flow through the recirculation system may be at 100 ml/minute, 200 ml/minute, or less.

It will be understood that numbers of flow and diameters can be adapted to a desired flow size for a given application.

In accordance with a method of installing the recirculation system 400, the water source connection 406, tool connection 414, the pump 424, and recirculation return tube 418 are connected to the sanitizing device 1, as described above.

The recirculation system 400 may reduce the number of pathogens per amount of fluid. For example, the recirculation system 400 may reduce the number of pathogens at the tool to below 200 pathogens/ml (as set by an ADA directive). The recirculation system 400 may reduce the number of pathogens without having to use environmentally dangerous chemicals or carcinogenic byproduct producing elements.

The recirculation system 400 may be particularly advantageous. Scientists in this field have been trying to solve the water biofilm problem over the last 40 years and have been determined to eliminate water tube biofilm and keep biofilm from occurring in order to reduce the pathogens/ml of water. The system 400 may reduce the pathogen counts below 200 pathogens/ml of water.

The recirculation system 400 may be applicable to medical applications where physical space is limited by using a single UV source to be able to disinfect compressed line air and pressurized city water source before utilizing in medical use.

In a particular embodiment, the recirculation system 400 is installed in a dental clinic where a user uses both water and compressed air, for example, for open mouth surgery and implants in less than 0.1 liter/minute. Biofilm may be a hazard based on water source contamination. For example, the ADA has found biofilm contamination that results in causalities, including death from contaminated water source used in dental clinics. A common water quality requirement is 500 pathogens/ml or less for any drinking water for healthy humans. While the ADA initially used the 500 pathogens/ml or less standard, as of year 2000, the ADA reduced the acceptable level of pathogens down to 200 pathogens/ml in order to prevent any further serious infections mainly for immune system suppressed patients.

Pathogens may be introduced into water in a number of ways. For example, the incoming water supply from the city and building pipes may be a contamination source. Further, biofilm may build up a colony of microorganisms as deadly pseudomonas inside the small diameter dental equipment water/air tubes. Biofilm is considered as a structured microbial community and a natural form of growth of most microorganisms. Biofilm is constructed by adhesion of the minerals, pathogens and related fibrin like substances using many adhesion processes as such as Van der Walls forces to form a matrix in the polymeric thin diameter water lines. Pseudomonas biofilm matrix is composed of various polymeric substances, polysccharides and proteins forming a significant part. The biofilm formation is often a very rapid process covering the inner surfaces of the tubing due to very high ratio of the wall surface to volume due to very thin tubing used in main controls of the dental equipment, drills, cleaning apparatus and like. Biofilm adhesion may begin in a few days and becomes difficult to remove from inside the flexible small diameter tubes mechanically or chemically. As water flows through the biofilm covered tube, the biofilm sheds slowly the pathogens and contaminating the water flow thereby, increasing the pathogen count per ml of water as such that the longer the water flows through the tubing the higher the pathogen count per ml of water.

Some approaches attempt to eradicate the biofilm by means of chemicals which may take up to a full day of filling the tubes. Some chemicals may work temporarily but usually each particular chemical eradicating either bacteria and viruses displaces the natural balance of the biofilm ecosystem and, as such, other fungus and related microorganisms may take over the colony and cause mutations in the colony. There is a resistance to using chemical disinfectants and antibiotics. Further, biofilms are relatively very resistant to mechanical destruction and eradication as biofilms may have a greater ability to effectively repair. Further, some chemicals react with organic material to produce carcinogenic by-products which may enter the mouth of the patient or in the air near the dental hygienist or the dentist with possible long term health effects.

Some scientists have tried ultrasonic waves, ozonation, nanoparticle silver compounds, and using bismuth antimicrobial compounds against Gram-positive and Gram-negative microorganisms including P. aeruginosa, heating the water, or drying the tubes overnight with compressed air. However, these methods may not provide the desired level of sanitization. Eradicating biofilm once established was not the goal but rather reducing the pathogen count below the desired threshold.

In some cases, dental water is run through the tool for at least 2 minutes every morning and preferably 8 minutes every morning and also every few hours during the day, the flow of water reduces the pathogen count below 200 pcs/ml if the user uses clean water to begin with through the water circuit. Unfortunately, dental clinics have not found it easy to do this while using existing equipment on a daily and hourly routine and at the same time to secure the quality of the water with any kind of measurement and alert system minute to minute against any malfunction. In some cases, a bottled water system filled with clean or sterilized water or with added germ killing solutions and tablets along with peroxide and similar new chemicals is energized with dental compressed air to pressurize the water in order to make it flow through the small diameter tubes to cool, clean and turn some of the hand tools used by the dentists. This system helps to eliminate the problems associated by the use of contaminated city water but may not remove or reduce the pathogens added to this water as it flows through the dental equipment circuitry where there is a prior biofilm development. Even for clean water supply with this bottled sample; it has been observed that the water bottle itself has a small bottle neck and difficult to clean and itself gets covered with a biofilm unless; pumped with dangerous chemicals every day to keep the microorganisms from colonizing on the inner surface.

Further, the bottle containing usually a liter of sterilized water may get pressurized with the unsanitary compressed air from usual dental compressor which automatically contaminates this clean water source. This so called clean water, gets contaminated by the pathogen colonies as it flows in a biofilm containing tube system down draft as the bottled sterilized water cannot be placed right prior to the dentist's hand piece because of the special pressure and flow adjustment controls and valves placed in the dental unit in order to control the flow in a special circuitry. Overall this system may not achieve the desired results either as the pathogen count goes up if the water flow stops even a few hours or overnight or on the weekends as the stagnant water in the system may quickly become contaminated. Accordingly, it may be desirable to use the recirculation system 400.

While the above description provides examples of one or more apparatus, methods, or systems, it will be appreciated that other apparatus, methods, or systems may be within the scope of the claims as interpreted by one of skill in the art.

1. A device for preventing build up and/or reducing biofilm pathogens from medical or dental fluid, the device comprising:
a housing defining a sanitizing chamber;
a water conduit extending through the sanitizing chamber, the water conduit channeling water from a water inlet to a water outlet;
a gas inlet for receiving pressurized gas;
a gas outlet, the pressurized gas flowing from the inlet to the gas outlet through the sanitizing chamber; and
a light source for emitting light into the sanitizing chamber, the emitted light being incident on the water conduit and the pressurized gas in the sanitizing chamber and having wavelengths effective for removing biofilm pathogens from the water and the pressurized gas to sanitize the water and the pressurized gas;
wherein the sanitized water and gas is used when performing medical or dental procedures.

2. The device of claim 1 further comprising a recirculation system comprising:
a recirculation return tube for recirculating water from the water outlet to the water inlet;
a water source connection fluidly connecting a water source and the recirculation return tube to the water inlet; and
a tool connection fluidly connecting a water control circuit outlet to a tool joint and the recirculation return tube, wherein the tool joint is connectable to a medical or dental surgical tool that uses the sanitized water and pressurized gas.

3. The device of claim 2 wherein the recirculation system further comprises:
a recirculation pump for pumping water in the recirculation return tube.

4. The device of claim 3, wherein the recirculation system further comprises:
a flow sensor to determine water flow in the recirculation return tube, wherein when the determined flow is below a predetermined level, the recirculation system will trigger an alarm.

5. The device of claim 3 wherein the recirculation system further comprises:
a one way check valve in the recirculation return tube to prevent backflow of water in the recirculation return tube.

6. The device of claim 1, wherein the pressurized gas remains pressurized while flowing through the sanitizing chamber.

7. The device of claim 1, wherein the water flowing through the water conduit is physically separated from the pressurized gas flowing through the sanitizing chamber.

8. The device of claim 1, wherein the sanitizing chamber is elongated and wherein the light source extends along a portion of a length of the elongated sanitizing chamber, the light being emitted in a radial direction of the sanitizing chamber.

9. The device of claim 1, wherein the water conduit is coiled about a first portion of the light source and wherein at least a second portion of the light source is free of the coiled water conduit.

10. The device of claim 9, wherein at least a quarter of the length of the light emitting source is free of the coiled water conduit.

11. The device of claim 1, further comprising at least one member disposed in the sanitizing chamber for causing turbulence in the pressurized gas within the sanitizing chamber.

12. The device of claim 11, wherein the at least one member comprises a baffle plate positioned in proximity of the gas inlet, the baffle plate defining one or more openings for causing the turbulence in the pressurized gas.

13. The device of claim 1, further comprising a condensation relief valve for releasing moisture from the sanitizing chamber.

14. The device of claim 1, further comprising a gas relief valve for releasing gas from the sanitizing chamber when the pressure in the sanitizing chamber rises above a predetermined pressure threshold.

15. The device of claim 1, wherein the housing comprises:
a plurality of upstanding walls defining a first end opening and a second end opening of the sanitizing chamber;
a first removable cap for sealing with a light support the first end opening of the upstanding walls, the light support further supporting the light source; and
a second removable cap for sealing the second end opening of the upstanding walls.

16. The device of claim 1, wherein the housing comprises:
a plurality of upstanding walls defining the sanitizing chamber, the upstanding walls being thermally conductive and further comprising at least one heat-sink member for dissipating heat from the sanitizing chamber.

17. The device of claim 1, further comprising:
an ultra-violet sensor for measuring an ultra-violet light level within the sanitizing chamber;
an alarm; and
a controller for receiving the measured ultra-violet light level and controlling the alarm to emit an alarm indicator when the measured ultra-violet light level falls below a predetermined ultra-violet light threshold.

18. The device of claim 1, wherein the sanitary chamber is sized based on an expected rate of use of the pressurized gas and a predetermined amount of exposure of pressurized gas to the emitted light and wherein the water conduit is sized based on an expected rate of use of the water and a predetermined amount of exposure of water to the emitted light.

19. A device for preventing build up and/or reducing biofilm pathogens from pressurized gas used in a medical or dental surgical tool, the device comprising:
a housing defining a sanitizing chamber;
a gas inlet for receiving pressurized gas;
a gas outlet, the pressurized gas flowing from the gas inlet to the gas outlet through the sanitizing chamber;
a light source for emitting light into the sanitizing chamber, the emitted light being incident on the pressurized gas in the sanitizing chamber and having wavelengths effective for removing biofilm pathogens from the pressurized gas to sanitize the water and the pressurized gas; and
at least one member disposed in the sanitizing chamber for causing turbulence in the pressurized gas within the sanitizing chamber
wherein the sanitized gas is used when performing medical or dental procedures.

20. A device for preventing build up and/or reducing biofilm pathogens from medical or dental fluid, the device comprising:
a housing defining a sanitizing chamber;
an inlet for receiving one of pressurized gas and water;
an outlet, said one of the pressurized gas and water flowing from the inlet to the outlet through the sanitizing chamber;
a light source for emitting light into the sanitizing chamber, the emitted light being incident on said one of the pressurized gas and water gas and having wavelengths effective for removing biofilm pathogens from said one of the pressurized gas and water to sanitize the water or the pressurized gas;
an ultra-violet sensor for measuring an ultra-violet light level within the sanitizing chamber;
an alarm; and
a controller for receiving the measured ultra-violet light level and controlling the alarm to emit an alarm indicator when the measured ultra-violet light level falls below a predetermined ultra-violet light threshold
wherein the sanitized water or gas is used when performing medical or dental procedures.
